(19) United States
(12) Patent Application Publication
(10) Pub. No.: US 2025/0341505 A1
(43) Pub. Date: Nov. 6, 2025

(54) PROCESS FOR CHECKING AND ADJUSTING A GAS SENSOR

(71) Applicant: Drägerwerk AG & Co. KGaA, Lübeck (DE)

(72) Inventors: Bernd-Michael DICKS, Lübeck (DE); Robert JAHNS, Lübeck (DE); Martin HAIDA, Lübeck (DE); Andreas SCHAUB, Lübeck (DE)

(21) Appl. No.: 19/196,848

(22) Filed: May 2, 2025

(30) Foreign Application Priority Data
May 3, 2024 (DE) 10 2024 112 477.6

(51) Int. Cl. G01N 33/00; G01N 21/3504

(52) U.S. Cl. CPC G01N 33/0072; G01N 21/3504

(57) ABSTRACT

A process (100) and system perform an adjustment/calibration on a gas sensor (60). The process makes it possible to correctly determine an offset (73) of the gas sensor (60) even with an unknown and non-zero interference gas concentration.

### CROSS REFERENCE TO RELATED APPLICATIONS

This application claims the benefit of priority under 35 U.S.C. § 119 of German Application 10 2024 112 477.6, filed May 3, 2024, the entire contents of which are incorporated herein by reference.

### TECHNICAL FIELD

The invention relates to a process for operating a gas sensor with verification and adjustment. The gas sensor to be tested is configured to determine the concentration of at least one gas component in a breathing gas mixture (respiratory gas mixture). Devices for determining the concentration of gas components in a breathing gas mixture are used, for example, to determine the concentration values of carbon dioxide exhaled by patients.

### BACKGROUND

DE 10 047 728 B4 describes a sensor for measuring carbon dioxide, nitrous oxide and anesthetic gases. In order to achieve high-quality measurements of gas concentrations, adjustments, calibrations, checks and balancing measures are usually carried out before and during measurement operation. Common calibration measures can be configured as zeroing.

During zeroing, the sensor or the sensory measuring elements and/or sensor electronics can be calibrated without the presence of a target gas (sample gas/measured gas). For this purpose, in conventional embodiments, the first step is to ensure that no quantities of sample gas are present in or on the sensor; this is usually achieved by purging (flushing) for a certain period of time with a purge gas (flushing gas), whereby the purge gas is selected in such a way that the sensor does not show any measuring reaction to this purge gas. In many cases, ambient air can be used as the purge gas for this purpose, which in conventional designs must flow through the sensor itself as well as the supply lines (hose lines), conveying (delivering) systems (pumps, valves) of the measuring system for a predetermined period of time so that it can be safely assumed that no residual amounts of sample gas can remain in the sensor or in the measuring system.

The measuring system is at least not ready to measure with full measuring accuracy for the time of the predetermined time period (duration) intended for flushing.

However, in some applications—in particular when monitoring breathing gas supply systems for patients on an anesthesia system or on a ventilation system with a breathing gas mixture-only a very brief interruption of measurement operation of less than 0.5 minutes is acceptable.

In many designs of measuring systems with supply lines (hose lines) and conveying systems (pumps, valves), residual quantities of anesthetic gases can adhere to the materials used and cannot be removed again during short flushing times of less than 0.5 minutes, so residual quantities can still desorb from the material of the hose lines during a zeroing procedure, reach the sensor and influence the result of the zeroing procedure.

### SUMMARY

It is an object of the invention, based on the aforementioned state of the art and the disadvantages described therein, to provide a process and sensor system for operating a gas sensor for determining the gas concentration of at least one gas component in a breathing gas mixture, which enables zeroing during ongoing measurement operation. The time required for zeroing should be such that there is no significant or only a very brief interruption in measurement operation as to measuring the sample gas (target gas).

The problem is solved, and the objects are attained by the features of features according to the invention.

The problem is solved, and the objects are attained by a process for operating a sensor with process features according to the invention and by a sensor system configured to operate a sensor with system features according to the invention.

Advantageous embodiments of the invention are disclosed herein, including in the following description, the figure and the claims.

The embodiments described each represent particular embodiments both individually and in combination or combinations with one another. All and possible further embodiments resulting from the combination or combinations of several embodiments and their advantages are nevertheless also covered by the inventive concept, even if not all possible combinations of embodiments are described in detail.

The terms and abbreviations used in the context of the present invention are summarized below and explained and defined in short form in each case.

Sample gas: A sample gas (measured gas-gas to be measured), often also referred to as a target gas, is a gas or a gas mixture of unknown gas composition with unknown concentrations of gas components, to which the gas sensor reacts with a sensor response depending on the concentrations of the individual gas components given at the time of measurement. The sensor response can be in the form of an electrical signal, for example.

Test gas: A test gas is a gas or gas mixture with a known gas composition with known concentrations of gas components to which the gas sensor reacts with a known and reproducible sensor response. The sensor response can be in the form of an electrical signal, for example.

Purge gas or a zeroing gas: A purge gas or a zeroing gas is a test gas of known gas composition and concentrations of gas components, to which the gas sensor reacts reproducibly with a zero signal as a sensor response. The sensor response can be in the form of an electrical signal, for example.

The zero signal indicates that no components above the detection limit (detection threshold) of the gas sensor are contained in the gas sample supplied to the gas sensor. This means that the number of molecules of the sample gas or the molar quantity of sample gas present in the gas sample is below the detection limit.

If an operating state with purge gas or zeroing gas results in a sensor response with a positive offset at the gas sensor, a certain number of molecules of the sample gas or another gas or gas mixture for which the gas sensor has a cross-sensitivity are present in the gas sample. If an operating state with a sample gas results in a sensor response with a negative offset at the gas sensor, zeroing was carried out in a situation in which a certain number of molecules of the sample gas were present in the gas sample during zeroing. This can be interpreted as an indication that no offset correction was applied when performing the zeroing.

Parameter set Px: A parameter set Px can include parameters determined during the calibration measures of the gas sensor, such as adjustments, calibrations, checks or zeroing. These include, for example, characteristic curves with curve shape, a gradient and an offset or interpolation values or data sets suitable for approximating characteristic curves.

First flow rate V1/dt and second flow rate V2/dt: A first flow rate V1/dt and a second flow rate V2/dt represent operating situations in which different quantities of a test gas are supplied to the gas sensor. Instead of the term flow rate V/dt, the terms “flow” or “mass flow” are often used.

Signal S1, time t1 and signal S2, time t2:

A signal S1 represents a sensor response at time t1.

A signal S2 represents a sensor response at time t2.

First time period Δta: A first time period Δta begins at time t1 and lasts until time t2. During the first time period Δta, an inflow to the gas sensor occurs with the first flow rate V1/d combined with a measured value acquisition of the signal S1.

Second time period Δtb: A second time period Δtb follows the first time period Δta and begins at time t2 in conjunction with a measured value acquisition of the signal S2. From time t2, the gas flows to the gas sensor at the second flow rate V2/dt for the time period of the second time period Δtb. After the time period of the second time period Δtb has elapsed, there is a transition to continuous measurement operation (continuous measurement mode) with the supply of sample gas to the gas sensor and continuous recording of further measured values Sn.

A process for checking and adjusting a gas sensor with a determination of a parameter set Px for an application of a measured value correction in continuous measuring operation of the gas sensor in a metrological detection of a sample gas can be configured according to the invention, as set out and described in the following manner:

After an initiation beginning with a start
activation of a switching unit to provide quantities of a test gas,
activation of a first operating state with a stable first flow state with a first flow rate V1/dt,
a measured value acquisition of signals S1 of the gas sensor at a first time t1,
activation of a second operating state with a stable second flow state with a second flow rate V2/dt,
a measured value acquisition of signals S2 of the gas sensor at a second point in time t2.

A parameter set Px is then determined on the basis of the signal S1, the signal S2, the first flow rate V1/dt and the second flow rate V2/dt

The procedure for checking and adjusting the gas sensor with determination of a parameter set Px can be carried out with measured value acquisition, activation and deactivation of operating states, preferably by a control unit.

After the procedure has been carried out, there is a transition to continuous measurement operation with acquiring/recording of further measured values Sn.

The first flow rate V1/dt and the second flow rate V2/dt are supplied to the gas sensor by means of—usually flexibly configured—hose lines. Such a hose line can, for example, be configured as a sample line, which enables a suction measurement with supply of gas quantities from a measurement location—for example directly from the mouth/nose area of a patient—to the gas sensor. Such a configuration is often referred to as a “side stream” measurement.

In a further embodiment, the gas sensor can be integrated into the supply and/or removal of breathing gases (respiratory gases) to the patient by means of a hose line. Such an arrangement is often referred to as a “main stream” measurement.

The hose lines are made of materials such as plastic materials, for example, which have the property of adsorbing and/or absorbing a certain number of molecules or molar quantities of gases—in particular the sample gas—when a sample gas flows through them. Such materials include silicone, polyurethane (PU) and fluororubber (FKM).

After a certain period of time or—in the case of a flow with a gas different from the sample gas, such as the test gas, purge gas or zeroing gas—possibly also within significantly shorter periods of time, this number of molecules or molar quantities of sample gas is released back into the gas mixture currently present in the hose line by means of desorption.

It is essential for the determination of the parameter set Px that the second flow rate V2/dt is formed with a flow rate that differs from the first flow rate V1/dt.

This advantageously enables desorption of residual quantities of sample gas from a hose line during the time period of the first flow rate V1/dt and during the time period of the second flow rate V2/dt, each with a different degree of dilution by the test gas, so that the number of molecules of sample gas which can enter the hose line during flushing (purging) with the first flow rate V1/dt and reach the gas sensor via the hose line is different from the number of molecules of sample gas which can enter the hose line during flushing with the second flow rate V2/dt and reach the gas sensor via the hose line.

The resulting different dilutions at two different flow rates V1/dt #V2/dt thus make it possible in an advantageous way to reduce the time required for zeroing compared to zeroing with almost complete flushing of residual components of molecules or molar quantities of the sample gas.

In a preferred embodiment, the parameter set Px can comprise an offset. If there is a difference between the two flow rates V1/dt; V2/dt, the offset in the parameter set Px can be determined using the following formula:

$$Offset = f ( S 1 , V 1 , S 2 , V 2 ) = \frac{S 1 * \frac{V 1}{dt} - S 2 * \frac{V 2}{dt}}{\frac{V 1}{dt} - \frac{V 2}{dt}} \qquad Formula 1$$

The parameter set Px thus represents an offset that results when carrying out a zeroing procedure in those cases in which different quantities of sample gas molecules reach the gas sensor at the time with the first flow rate V1/dt compared to the time with the second flow rate V2/dt.

If a significantly longer flushing with a purge gas were carried out and the desorption rate therefore tends towards zero, there would be no signal difference between the signals S1 and S2 and therefore—as an application of the above formula 1 with corresponding values for S1 and S2 also shows—the offset would then also tend towards zero. Explanations of the physical background of Formula 1 can be found at the end of the description of FIG. 1.

In a preferred embodiment, the parameter set Px can be used in a continuous measurement operation. As soon as the offset Px is determined, the sample gas can be fed back to the gas sensor and continuous measurement operation can be performed

The offset Px is used to correct the measurement signal Sn in continuous measurement operation—for example according to the relationship S′n=f (Sn; Px)—and thus obtain a corrected measurement signal S′n. The above relationship can be formed in the simplest way as follows: S′n=Sn+Px and can be taken into account by the control unit when operating the gas sensor during measured value acquisition, measured value processing and/or measured value provision.

In a further preferred embodiment, the parameter set Px can indicate a faulty state (defective state) of the gas sensor and/or the switching unit. If the parameter set Px indicates the faulty state, the measurement accuracy may be reduced in continuous measurement operation.

In the event of a faulty state of the gas sensor and/or switching unit, the process according to the invention can be carried out repeatedly in a further preferred embodiment.

In these further preferred embodiments, a change to continuous measurement operation can be made by deactivating the provision of quantities of the test gas and providing quantities of the sample gas.

In a further preferred embodiment, the sample gas and the test gas can be supplied to the gas sensor in a side stream as a gas supply by means of an suction measurement, preferably by means of a gas conveying unit P and a sample gas line (sample line).

In a further preferred embodiment, the sample gas and the test gas can be measured by means of a measurement in a main gas stream.

In a further preferred embodiment, the activation of the first operating state can be delayed for a delay time T_D1 following the activation of the provision of quantities of the test gas.

In a further preferred embodiment, the measured value acquisition of signals S1 of the gas sensor can be delayed at the first time t1 after a waiting time T_D2.

In a further preferred embodiment, the measured value acquisition of signals S2 of the gas sensor can be delayed and carried out at the second time t2 after a waiting time T_D3.

The delay time T_D1, the waiting times T_D2 and T_D3 can be useful to wait for a period of transient or switchover processes between sample gas and test gas or periods of time with effects of transient or overshoot, which can occur with changes in the flow rates, before recording measured values or signals.

In a further preferred embodiment, the two flow rates V1/dt and V2/dt can differ by at least a factor in the range from 1.5 to 3.

In a further preferred embodiment, one of the two flow rates V1/dt or V2/dt can be selected in such a way that a flow-free state is given at the gas sensor.

In a further preferred embodiment, the delay time T_D1 can be selected as a function of a response time of a valve unit in the switching unit.

In a further preferred embodiment, the first waiting time T_D2 and/or the second waiting time T_D3 and/or the delay time T_D1 can be selected as a function of properties of the gas conveying unit such as conveying rate, start-up response time, control behavior. The gas conveying unit can be configured as a pump, preferably as an electromechanical pump, a piezoelectric pump.

In a further preferred embodiment, the delay time T_D1 can be selected as a function of a response time of a valve unit in the switching unit.

In a further preferred embodiment, the first waiting time T_D2 and/or the second waiting time T_D3 can be selected as a function of the diameter, length, volume and/or material of a hose line. The sample gas line can be configured as a hose line, in particular as a flexible hose line. Suitable materials for a flexible hose line are, for example, silicone, polyurethane (PU), fluororubber (FKM).

The present invention will be explained in more detail in the description below with reference to the drawing, without limiting the general concept of the invention. The various features of novelty which characterize the invention are pointed out with particularity in the claims annexed to and forming a part of this disclosure. For a better understanding of the invention, its operating advantages and specific objects attained by its uses, reference is made to the accompanying drawing and descriptive matter in which preferred embodiments of the invention are illustrated.

### BRIEF DESCRIPTION OF THE DRAWING

In the drawing:

FIG. 1 is a schematic view showing features of a sensor system with a sequence for checking and/or adjusting a gas sensor;

### DESCRIPTION OF PREFERRED EMBODIMENTS

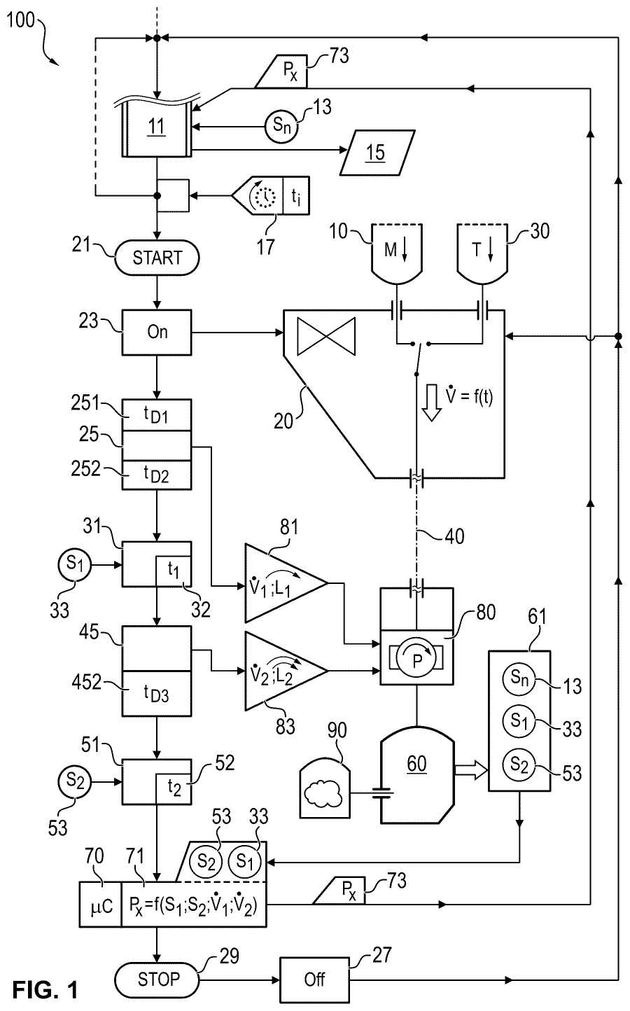

Referring to the drawings, FIG. 1 shows a sequence 100 for checking and/or adjusting a gas sensor 60. FIG. 1 also shows gas sensor system features including the gas sensor 60. In the sequence 100, a change is made from a continuous measuring mode 11 with detection of measured values Sn 13 of a gas sensor 60 and their provision 15 to a sequence for calibrating the gas sensor 60. In continuous measuring mode 11 with detection of measured values Sn 13 of the gas sensor 60, quantities of a sample gas 10 are continuously supplied to the gas sensor 60 by means of a gas conveying unit 80 (pump)—outlined as an example in this FIG. 1—and a hose line (sample line), which is usually of flexible design, and are led away again from the gas sensor 60 by means of a gas scavenging (outlet) line 90

The sequence 100 can be coordinated, for example, by a control unit 70, which is configured by means of suitable components (μC, RAM, ROM, program code) to control a mixing or switching unit 20—presented/outlined as an example in this FIG. 1—and thus enable the gas sensor 60 to be calibrated.

Furthermore, the control unit 70 is configured to acquire/record, process and evaluate signals 61 from the gas sensor 60 by measurement.

One possible evaluation for an optical gas sensor is, for example, an evaluation in relation to an attenuation of infrared-optical radiation as a function of a sample gas in a gas sample 10, for example in the form of a proportion of the sample gas (the gas of interest) in the gas sample 10, such as a volume concentration or a partial pressure and its provision 15 and/or its output 15, for example on an output unit (display).

Other possible gas sensors that can be checked with the sequence 100 can be based on electrochemical, electroacoustic, acoustic (ultrasonic), paramagnetic or catalytic measuring principles, for example.

The change from the continuous measuring mode 11 to the start 21 of the sequence for calibration takes place in the illustration according to this FIG. 1 by a request 17, which takes place, for example, externally—triggered by a user activity—or triggered by a time event 17, as shown as an example in this FIG. 1. After the start 21, the switching unit 20 is activated 23, which is configured—for example with a valve arrangement—to switch between the sample gas 10 and a test gas 30 in order to perform an adjustment of the gas sensor 60, for example for a calibration, in particular for a zero point calibration or offset adjustment.

After activation 23 of the switching unit 20 a stable first operating state 25 with a first flow state 81 of the gas conveying unit (pump P) 80 with a conveyance of test gas 30, which is supplied to the gas sensor 60 at a first flow rate V1/dt 81, results at a first time t1 32—possibly after an optional certain delay time 251, which may be caused by changeover processes of the valve arrangement in the switching unit 20. The first flow rate V1/dt supplied to the gas sensor 60 can preferably be configured as a flow rate in the range from 0.1*10⁻³ liters/minute to 100*10⁻³ liters/minute.

Then, after a certain waiting time 252 with a predetermined time period, which is determined by a minimum time period required for a flow of quantities of test gas 30 through the gas sensor 60, a measured value acquisition 31 of signals S1 33 during the first flow state 25 and their provision to the control unit 70 takes place.

After the measured value acquisition 31, a stable second operating state 45 with a second flow state 83 of the gas conveying unit (pump P) 80 is set at a second time t2 52, wherein a conveyance of test gas 30 with a second flow rate V2/dt 83 is supplied to the gas sensor 60. The second flow rate V2/dt 83 supplied to the gas sensor 60 can preferably be configured as a flow rate in the range from 0.1*10⁻³ liters/minute to 200*10⁻³ liters/minute.

In alternative embodiments of gas sensors, the first flow rate V1/dt 81 can be configured as a flow rate of 0.00 liters/minute. In further alternative embodiments of gas sensors, the second flow rate V2/dt 83 can be configured as a flow rate in the range from 1.0 liter/minute to 15.0 liters/minute, for example also 4.0-5.0 liters/minute.

The first flow rate V1/dt 81 and the second flow rate V2/dt 83 are clearly different from each other, i.e. they are configured as different flow rates V1/dt 81, V2/dt 83, which flow through the same hose system. A ratio of the flow rates V1/dt 81, V2/dt 83 to each other of 1:2 up to 1:100 is a practicable range.

As an approximate orientation of the flow ranges of the two flow rates V1/dt 81 and V2/dt 83, for example, it can be assumed that the gas supply with the test gas 30 is selected to be approximately 50% lower than the gas supply with the sample gas 10, for example V1/dt=4.0 liters/minute and V2/dt=8.0 liters/minute.

In particular embodiments, one of the two flow rates V1/dt 81 or V2/dt 83 can be configured as a very low flow rate or also as an almost flow-free state (V/dt˜0) or as a flow-free state (V/dt=0).

In special embodiments, one of the two flow rates V1/dt 81 or V2/dt 83 can be configured as a flow-free state (V/dt=0) and the other of the flow rates V1/dt 81 or V2/dt 83 can be configured in the range from 1.0 liter/minute to 15.0 liters/minute.

A measured value acquisition 51 of signals S2 53 then takes place during the second flow state 45 and their provision to the control unit 70.

Based on the signals S1 33 and the signals S2 53 of the gas sensor 60, which were measured for two different flow states V1/dt 81 and V2/dt 83 effective in the hose line 45, a parameter set Px 73 is determined by the control unit 71 in the subsequent step 71, the sequence for calibration comes to an end (stop) 29, and the continuous measuring operation 11 is resumed with deactivation 27 of the gas switchover 20 with a change to a supply of sample gas 10 to the gas sensor 60.

This parameter set Px 73 is used when resuming continuous measuring operation 11 in order to eliminate as far as possible any effects that occur during measuring operation 11 due to molecules of the sample gas penetrating the material of the hose line 40 (absorption) or adhering to or on the inner walls of the hose line 40 (adsorption). These molecules of sample gas 10 in the hose line are released again when the flow passes through the hose line 40 (desorption).

If a test gas 30, which does not contain any components of the desired sample gas (sample gas of interest) or concentrations of an interfering gas, flows through the gas sensor 60, the gas sensor 60 only shows a signal that is >99% unaffected by the sample gas or the interfering gas after a very long purge time of more than 240 seconds. Such long purge times are necessary due to the release (desorption) of molecules of the sample gas 10 from the hose line 45 into the freshly supplied test gas 30 in order to avoid almost any effect on a subsequent zero-point calibration. However, such long flushing times are not possible in anesthesia applications, as continuous monitoring of concentrations of anesthetic gases in a patient's breathing gas mixture is required. Continuous monitoring must not be interrupted by long pauses, which occur during regular zero-point calibration with a test gas 30.

Regular zero-point calibrations of the gas sensor 60 with a test gas 30 result from the requirements for measurement accuracy in anesthesia applications and serve to eliminate effects that can influence the properties of the gas sensor 60, In the case of an infrared optical gas sensor, these include, for example, contamination effects in the sensor cuvette, ageing effects of the radiation source and detectors, ageing and drift effects in the measurement electronics, temperature fluctuations that continuously affect the detectors and measurement electronics with signal amplification and signal filtering during operation of a gas sensor.

The methodology shown in the sequence 100 uses the signals S1 33 and S2 53, associated the flow states V1/dt 81 and V2/dt 83 prevailing in the hose line 40, to estimate a signal with a flow through the hose line 40 with the test gas 30 for less than 20 seconds, which is almost identical to a zero signal with sufficiently long flushing of the hose line 40 and the gas sensor 60, and then to use this for the continuation of the continuous measuring operation 11.

In this way, zero-point calibrations of short time periods of less than 40 seconds, for example 10 seconds to 30 seconds, can be carried out regularly from time to time—for example hourly or even daily—during ongoing measurement operation and used as parameter set Px 73 in the signal evaluation in order to ensure very precise measurement accuracy of the gas sensor continuously over longer operating and usage times. Since the same test gas 30 is supplied at two different times t1 32 and t2 52 with different flow velocities (flow rates) V1/dt 81 and V2/dt 83, a zero point error to be corrected or a sensor offset can be determined from the knowledge of flow velocities V1/dt 81 and V2/dt 83 as well as the recorded sensor signals S1 51, S2 53 even if the concentration of an interfering gas is unknown during zero point calibration—often also referred to as zeroing—and used as parameter set Px 73 in continuous measurement operation. This makes use of the effect that the first flow rate V1/dt 81 and the second flow rate V2/dt 83 are implemented as two flow rates of different sizes (magnitudes), which however flow through the same hose line 40, but thereby release different quantities or a different number of molecules of the interfering gas from the material of the hose line 40 into flowing quantities of test gas 30.

In simple terms, the greater the flow rate V/dt [m/s²] and thus the flow velocity v [m/s] in the hose line, the fewer molecules of interfering gas are released from the material into the flowing gas volume and thus reach the gas sensor 60, where they affect the measurement signals S1 33, S2 53. Assuming that the two flow rates V1/dt 81 and/or the second flow rate V2/dt 83 and thus also the two flow velocities v1, v2 resulting in the hose line are significantly greater than the flow velocity of the interfering gas based on the number of molecules, which are released from the hose line 45 into the flowing gas quantities, the following relationship results for estimating the zero point error to be corrected as offset Px 73, as it can be determined during the evaluation 71 by the control unit 70 according to the formula 1 already mentioned in the general description:

$$Offset = f ( S 1 , V 1 , S 2 , V 2 ) = \frac{S 1 * \frac{V 1}{dt} - S 2 * \frac{V 2}{dt}}{\frac{V 1}{dt} - \frac{V 2}{dt}} \qquad Formula 1$$

Some aspects of the physical background and interrelationships behind Formula 1 are briefly explained below. To simplify matters, it is assumed that the gas sensor is supplied with ideal gases and that the general gas equation

$$p \cdot V = n \cdot R \cdot T , orn = \frac{p}{R \cdot T} \cdot V ,$$

can be applied with:
p as pressure in mbar, V as volume in ml or cm³, n as amount of substance in mol,

$$R = 83.14472 \frac{mbar \cdot l}{mol \cdot K}$$

as the universal gas constant. Converted into a mole flown in mol/min and volume flow \dot{V} in ml/min

$$\dot{n} = \frac{p}{R \cdot T} \cdot \dot{V} ;$$

Further conversion results in a molar mass in from the mole flow \dot{n}
as well as a mass flow \dot{m} in g/min:

$$\dot{m} = M \cdot \dot{n} = \frac{M \cdot p}{R \cdot T} \cdot \dot{V} = \frac{p}{{R}_{S} \cdot T} \cdot \dot{V}$$

with

$${R}_{S} = R / M ( in \frac{mbar \cdot l}{g \cdot K} )$$

as the specific gas constant.

In the situation that during zeroing with a zeroing gas (test gas) from the hose line material a certain number of molecules or a molar quantity of a previously accumulated gas, hereinafter referred to as “interfering gas”, re-enters the hose line by desorption, this results in a mole flow \dot{n}_{interfering} of interfering gas, which is additive to the pier flow \dot{n}_{zero} of zeroing gas through the hose line as gas flow \dot{n}_{zero}+\dot{n}_{interference} flows towards the gas sensor. Dalton's law applies to the mixture of zeroing gas and interfering gas in a gas mixture for the partial pressure sum in the gas mixture with:

$${p}_{ges} = \sum {p}_{i}$$

For the mole fraction yᵢ, which relates the molar amount of substance nᵢ of component i to the amount of substance n, the following applies accordingly:

$$\sum {y}_{i} = \sum \frac{{n}_{i}}{n} = 1.$$

The mole fraction y_{interfering} of the interfering gas as a function of the mole flow \dot{n}_{zero} of the zeroing gas and the mole flow \dot{n}_{stör} of the interfering gas is derived from the relationships described above:

$${y}_{interfering} = \frac{{\dot{n}}_{interfering}}{{\dot{n}}_{Zero} + {\dot{n}}_{interfering}}$$

The volume fraction r_{interfering} as a function of the zero gas flow \dot{V}_{zero} or interfering gas flow \dot{V}_{interfering} is derived from the relationships described above:

$${r}_{interfering} = \frac{{\dot{V}}_{interfering}}{{\dot{V}}_{Zero} + {\dot{V}}_{interfering}} .$$

Multiplying the volume percentage of the interfering gas by 100% by volume gives the sensor response of the gas sensor in % by volume:

$$Conc . Gas = {r}_{interfering} * 100 Vol . \%$$

Zeroing the gas sensor now serves to eliminate an unknown offset (Offs. In Vol. %) during the zeroing process. With an offset, the sensor response S of the gas sensor applies:

$$S = Conc . Gas + Offs . = {r}_{interfering} * 100 Vol . \% + Offs . = {y}_{interfering} * 100 Vol . \% + Offs .$$

If the interfering gas concentration were y_{interfering}*100 Vol. %=0, the offset could easily be determined and eliminated by a measurement without a corresponding target gas, resulting in: S=offs.

This results in the task of determining and eliminating this offset approximately correctly for an unknown interference gas concentration y_{interfering}*100 Vol. %≠0.

Two points in time t₁ and t₂ are to be considered here, whereby only the mole flow or volume flow of the zeroing gas is to change at these times.

The molar flow \dot{n}_{interfering} or volume flow \dot{V}_{interfering} of the interfering gas is assumed to be largely constant over the period of zeroing, but changes could be determined by longer measurements at times t₁ and t₂.

At time t1, this results in the mole flow \dot{n}_{zero,t1} or volume flow \dot{V}_{zero,t1} and at time t2 the mole flow \dot{n}_{zero,t2} or volume flow \dot{V}_{zero,t2}.

Both flows are different from each other, which is realized by the different flow rates (V1/dt 81; V2/dt 83).

It then follows from the above equation for time t1:

$${S}_{t 1} = Conc . {Gas}_{t 1} + Offs . = \frac{{\dot{V}}_{interfering}}{{\dot{V}}_{Zero , t 1} + {\dot{V}}_{interfering}} \cdot 100 Vol . \% + Offs .$$

And for the time t2:

$${S}_{t 2} = Conc . {Gas}_{t 2} + Offs . = \frac{{\dot{V}}_{interfering}}{{\dot{V}}_{Zero , t 2} + {\dot{V}}_{interfering}} \cdot 100 Vol . \% + Offs .$$

Equating via \dot{V}_{interfering} results in

$$\frac{{S}_{t 1} - Offs .}{100 Vol . \%} \cdot ( {\dot{V}}_{Zero , t 1} + {\dot{V}}_{interfering} ) = \frac{{S}_{t 2} - Offs .}{100 Vol . \%} \cdot ( {\dot{V}}_{Zero , t 2} + {\dot{V}}_{interfering} )$$

This results in the following by switching to Offs:

$$Offs . = \frac{{S}_{t 1} \cdot ( {\dot{V}}_{Zero , t 1} + {\dot{V}}_{interfering} ) - {S}_{t 2} \cdot ( {\dot{V}}_{Zero , t 2} + {\dot{V}}_{interfering} )}{( {\dot{V}}_{Zero , t 1} + {\dot{V}}_{interfering} ) - ( {\dot{V}}_{Zero , t 2} + {\dot{V}}_{interfering} )}$$

If \dot{V}_{zero,t1}>>\dot{V}_{interfering} and \dot{V}_{zero,t2}>>\dot{V}_{interfering} the above equation is simplified to:

$$Offs . = \frac{{S}_{t 1} \cdot {\dot{V}}_{Zero , t 1} - {S}_{t 2} \cdot {\dot{V}}_{Zero , t 2}}{( {\dot{V}}_{Zero , t 1} - {\dot{V}}_{Zero , t 2} )} .$$

While specific embodiments of the invention have been shown and described in detail to illustrate the application of the principles of the invention, it will be understood that the invention may be embodied otherwise without departing from such principles.

### REFERENCE NUMBERS LIST

10 Sample gas supply, sample gas supply, sample gas, target gas
11 Continuous measuring operation with sample gas
13 Signals Sn in continuous measurement operation
15 Provision of the measurement signals, output
17 Request/initialization for calibration, event (trigger event)
20 Mixing or switching unit (valve), sample gas/test gas
21 Start of the calibration process
23 Activation of gas switching
25 First operating state of the calibration, first flow state
27 Deactivating gas switching (switching back to sample gas supply)
29 End (stop) of the calibration process
30 Test gas supply, calibration gas
31 Measurement signal acquisition
32 First time t1
33 Signal S1 in calibration with the first flow state
45 Second operating state of the calibration, second flow state
40 Hose line with material, diameter, length, properties (gas type-spec. desorption, absorption, adsorption)
51 Measurement signal acquisition
52 Second time t2
53 Signal S2 in calibration with the second flow state
60 Gas sensor
61 Sensor data
70 Control unit
71 Evaluation, parameter determination
73 Parameter set Px (calibration, offset, slope)
80 Gas conveying unit, pump P
81 First flow condition, flow rate V1/dt with test gas
83 Second flow condition, flow rate V2/dt with test gas
90 Gas scavenging outlet (to environment, return to the circuit system in the anesthesia machine)
100 Procedure
251 Delay time after valve switchover to test gas
252 Waiting time after activation of the first flow state
452 Waiting time after activation of the second flow state

What is claimed is:

1. A process for checking and adjusting a gas sensor, the process comprising:
activating a switching unit to provide quantities of a test gas;
activating a first operating state with a stable first flow state of the test gas with a first flow rate of the test gas;
acquiring a first time measured value signal of the gas sensor at a first time, which is during the activated first operating state;
activating a second operating state with a stable second flow state of the test gas with a second flow rate of the test gas, wherein the second flow rate is different from the first flow rate;
acquiring a second time measured value signal of the gas sensor at a second time, which is during the activated second operating state; and
determining a parameter set based on the first time measured value signal, the first flow rate, the second time measured value signal, and the second flow rate, wherein the determined parameter set is configured as a correction of measured values acquired during a continuous measuring operation of the gas sensor, which measuring operation includes a metrological detection of a sample gas.

2. A process according to claim 1, wherein the parameter set is configured to correct the measured values of continuously acquired measurement signals during the continuous measurement operation, during which a gas measurement is carried out with the sample gas, and wherein the parameter set comprises a corrected offset of the measured values.

3. A process according to claim 2,
wherein the parameter set indicates a faulty state of the gas sensor and/or a faulty state of the switching unit, and
wherein for the continuously acquired measurement signal, detected in the continuous maturement operation, a reduced measuring accuracy is assigned during the continuous maturement operation, if the parameter set indicates the faulty state.

4. A process according to claim 3, wherein in the event of a faulty state of the gas sensor and/or switching unit, the process is carried out repeatedly.

5. A process according to claim 1, wherein, following the determination of the parameter set, the process comprises activating the provision of quantities of the test gas to the gas sensor and providing quantities of the sample gas.

6. A process according to claim 1,
wherein for the metrological detection of the sample gas and the test gas, the gas sensor is supplied with a gas sample by a sample gas line as a bypass gas stream by means of a suction measurement; or
wherein the sample gas and the test gas are measured by metrological detection in a main gas flow.

7. A process according to claim 1,
wherein the activation of the first operating state is delayed following activating the switching unit to provide quantities of a test gas for a settling time period; and/or
wherein the acquisition of measured value signals of the gas sensor is delayed at the first time for a waiting time, and/or
wherein the acquisition of measured value signals of the gas sensor is delayed at the second time for a waiting time.

8. A process according to claim 1, wherein the first flow rate differs from the second flow rate by at least a factor in the range from 1.5 to 3.

9. A process according to claim 1, wherein one of the first flow rate and the second flow rate is selected such that a flow-free state is established at the gas sensor.

10. A process according to claim 7,
wherein the settling time period is selected as a function of a time response of a valve unit of the switching unit, and/or
wherein the first waiting time and/or the second waiting time and/or the settling time period are selected as a function of properties of a gas conveying unit, the properties comprising at least one of conveying capacity, start-up response time, control behavior.

11. A process according to claim 7,
wherein the settling time period is selected as a function of a response time of a valve unit of the switching unit, and/or
wherein the first waiting time and/or the second waiting time are selected as a function of the diameter, length, volume and/or material of a hose line.

12. A process for checking and adjusting a gas sensor, the process comprising:
operating the sensor in a continuous measuring operation mode, which measuring operation mode includes a metrological detection of a sample gas and a generation of a measured value signal;
starting a checking and adjusting sequence comprising:
activating a switching unit to provide quantities of a test gas;
activating a first operating state of the sequence comprising a stable first flow state of the test gas to the sensor with a first flow rate of the test gas;
acquiring a first time measured value signal of the gas sensor at a first time, which is during the activated first operating state;
activating a second operating state of the sequence comprising a stable second flow state of the test gas to the sensor with a second flow rate of the test gas, wherein the second flow rate is different from the first flow rate;
acquiring a second time measured value signal of the gas sensor at a second time, which is during the activated second operating state;
determining a parameter set based on the first time measured value signal, the first flow rate, the second time measured value signal, and the second flow rate, wherein the determined parameter set is configured as a correction of measured values acquired during the continuous measuring operation of the gas sensor in the continuous measuring operation mode; and
correcting the measured value signal of the sensor with the parameter set.

13. A process according to claim 12,
wherein the switching unit comprises a valve unit configured to switch between the test gas and the sample gas; and
wherein the activation of the first operating state is delayed following activating the switching unit to provide quantities of a test gas for a settling time period; and/or the acquisition of measured value signals of the gas sensor is delayed at the first time for a waiting time, and/or the acquisition of measured value signals of the gas sensor is delayed at the second time for a waiting time.

14. A process according to claim 13,
wherein the stable flow state of each operating state is established by a conveying unit; and
wherein the settling time period is selected as a function of a time response of the valve; and/or the first waiting time and/or the second waiting time and/or the settling time period are selected as a function of properties of the gas conveying unit, the properties comprising at least one of conveying capacity, start-up response time, control behavior.

15. A process according to claim 13,
wherein the settling time period is selected as a function of a response time of a valve unit of the switching unit, and/or
wherein the first waiting time and/or the second waiting time are selected as a function of the diameter, length, volume and/or material of a hose line.

16. A sensor system comprising:
a gas sensor;
conveying unit configured to convey gas to the gas sensor;
a switching unit configured to switch between a sample gas supply and a target gas supply to supply either a sample gas or a target gas for conveyance to the sensor; and
a control unit operatively connected to the conveying unit, the gas sensor and the switching unit and configured:
to operate the sensor system with the sensor in a continuous measuring operation mode and with the switching unit switched to supply the sample gas or with the switching unit switched to supply the test gas to be conveyed by the conveying unit to the sensor, wherein the measuring operation mode comprises a metrological detection of the sample gas and a generation of a measured value signal; and
to operate the sensor system with a checking and adjusting sequence with the sensor in the continuous measuring operation mode, the sequence comprising:
activating the switching unit to supply the test gas;
activating a first operating state comprising a stable first flow state of the test gas to the sensor with a first flow rate of the test gas;
acquiring a first time measured value signal of the gas sensor at a first time, which is during the activated first operating state;
activating a second operating state of the sequence comprising a stable second flow state of the test gas to the sensor with a second flow rate of the test gas, wherein the second flow rate is different from the first flow rate;
acquiring a second time measured value signal of the gas sensor at a second time, which is during the activated second operating state;
determining a parameter set based on the first time measured value signal, the first flow rate, the second time measured value signal, and the second flow rate, wherein the determined parameter set is configured as a correction of measured values acquired during a continuous measuring operation of the gas sensor in the continuous measuring operation mode; and
correcting the measured value signal of the sensor with the parameter set.

17. A sensor system according to claim 16,
wherein the control unit is configured to detect that the parameter set indicates a faulty state of the gas sensor and/or a faulty state of the switching unit, and
wherein for the continuously acquired measurement signal, detected in the continuous maturement operation, a reduced measuring accuracy is assigned by the control unit during the continuous maturement operation, if the parameter set indicates the faulty state.

18. A sensor system according to claim 16,
wherein the switching unit comprises a valve unit configured to switch between the test gas and the sample gas; and
wherein the activation of the first operating state is delayed following activating the switching unit to provide quantities of a test gas for a settling time period; and/or the acquisition of measured value signals of the gas sensor is delayed at the first time for a waiting time, and/or the acquisition of measured value signals of the gas sensor is delayed at the second time for a waiting time.

19. A sensor system according to claim 18, wherein the settling time period is selected as a function of a time response of the valve; and/or the first waiting time and/or the second waiting time and/or the settling time period are selected as a function of properties of the gas conveying unit, the properties comprising at least one of conveying capacity, start-up response time, control behavior.

20. A sensor system according to claim 18,
wherein the settling time period is selected as a function of a response time of a valve unit of the switching unit, and/or
wherein the first waiting time and/or the second waiting time are selected as a function of the diameter, length, volume and/or material of a hose line.
